(19) United States
(12) Patent Application Publication
(10) Pub. No.: US 2020/0163955 A1
(43) Pub. Date: May 28, 2020

(54) STERILE COMPOSITIONS OF INDACATEROL SUITABLE FOR NEBULIZATION

(71) Applicant: GLENMARK SPECIALTY S.A., La Chaux-de-Fonds (CH)

(72) Inventors: Ulhas R. Dhuppad, Nashik (IN); Sunil Chaudhari, Nashik (IN); Suresh Rajurkar, Nashik (IN); Chetan Yewale, Dhule (IN)

(21) Appl. No.: 16/692,237

(22) Filed: Nov. 22, 2019

(30) Foreign Application Priority Data
Nov. 22, 2018 (IN) 201821044005

(51) Int. Cl. A61K 31/473; A61K 47/02; A61K 47/14; A61K 9/00

(52) U.S. Cl. CPC A61K 31/473; A61K 47/02; A61P 11/08; A61K 9/0073; A61K 47/14

(57) ABSTRACT

The present invention relates to a sterile pharmaceutical composition comprising indacaterol or a pharmaceutically acceptable salt thereof, for inhalation via nebulization to a subject in need thereof. The present invention also relates to a process of preparing such a pharmaceutical composition and the use of such compositions in the treatment of respiratory diseases.

### CROSS REFERENCE TO RELATED APPLICATIONS

This application claims priority to Indian Provisional Patent Application No. 201821044005 filed on Nov. 22, 2018, the entire contents of which are incorporated by reference herein.

### FIELD OF THE INVENTION

The present invention relates to a sterile pharmaceutical composition comprising indacaterol or a pharmaceutically acceptable salt thereof, for inhalation via nebulization to a subject in need thereof. The present invention also relates to a process of preparing such a pharmaceutical composition and the use of such compositions in the treatment of respiratory diseases.

### BACKGROUND OF THE INVENTION

Respiratory disorders include a number of airway diseases. Asthma and chronic obstructive pulmonary disease (COPD) are among the most prevalent and life-threatening conditions.

COPD is a chronic disorder that is characterized by loss of elasticity of the airways and air sacs, destruction of alveolar walls, inflammation of airways, and high mucus production in the airways. All of these effects lead to clogging of the airways making it difficult for the patient to breathe. Asthma, on the other hand, is a chronic disease involving airways of the lung that is characterized by coughing, wheezing, and shortness of breath.

Administration by inhalation is one route for the treatment of respiratory disorders. A number of medications delivered by inhalation include corticosteroids, beta-agonists, anticholinergic agents, and expectorants. Different dosage forms are available which include nebulization, dry powder inhalers, and metered dose inhalers.

Nebulization is one mode of delivery by inhalation and can be used to deliver medications across all age groups especially pediatric and geriatric population. The nebulization dosage form, unlike the dry powder and the metered dose inhalers, does not require synchronization with the user's breathing pattern to maximize the delivery.

Medicaments typically used for the treatment of respiratory diseases like COPD and asthma include beta-adrenoreceptor agonists, anti-muscarinic agents and corticosteroids. There is also a new category of active agents, ultra-long acting beta adrenoreceptor agonists (ultra LABA) which act typically for about 24 hours and provide the ease of once daily dosing. A newer ultra LABA in this class is indacaterol.

Indacaterol, 5-[(1R)-2-[(5,6-diethyl-2,3-dihydro-1H-inden-2-yl)amino]-1-hydroxyethyl]-8-hydroxy-1H-quinolin-2-one, and pharmaceutically acceptable salts thereof, are described in U.S. Pat. No. 6,878,721. Indacaterol for the treatment for COPD is described in U.S. Pat. No. 8,067,437.

The structure of indacaterol maleate, 5-[2-(5,6-Diethylindan-2-ylamino)-1-hydroxyethyl]-8-hydroxy-1H-quinolin-2-one maleate, is shown below:

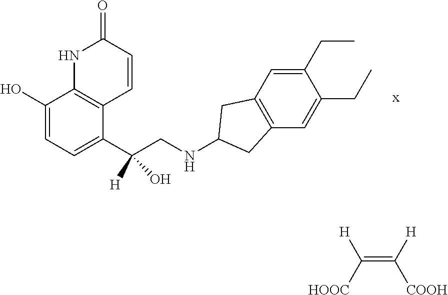 [SMILES: CCc1cc2c(cc1CC)CC(NC[C@H](O)c1ccc(O)c3[nH]c(=O)ccc13)C2 ; O=C(O)/C=C\C(=O)O]

Indacaterol is currently approved in U.S. as a dry powder inhaler as Arcapta Neohaler® and as a dry powder inhaler in combination with glycopyrrolate as Utibron®. U.S. Pat. No. 8,796,307 discloses a dry powder of indacaterol for inhalation. Indacaterol is characterized by low aqueous solubility and is classified as “very slightly soluble in water”. This poses a serious challenge to solubilizing indacaterol in order to formulate a nebulization dosage form. It would be advantageous to combine the advantage of a nebulization dosage form and the pharmacological properties of an ultra LABA such as indacaterol. However, considering the limited aqueous solubility of indacaterol, it still remains a challenge.

Chinese Patent Application No. CN 103860463 discloses indacaterol maleate solutions for use in inhalation sprays.

There exists therefore an unmet need in the art to effectively deliver indacaterol in nebulized form for the treatment of respiratory disorders.

### SUMMARY OF THE INVENTION

The present invention provides a nebulization composition comprising indacaterol or a pharmaceutically acceptable salt thereof.

In one aspect, the present invention relates to a sterile pharmaceutical composition for inhalation via nebulization to a subject. The nebulization composition comprises indacaterol or a salt thereof (e.g., a pharmaceutically acceptable salt thereof) and water. The pharmaceutical composition may be a solution or a suspension. The nebulization composition may be contained within a pre-filled container. The nebulization composition may be administered to relieve a subject suffering from a respiratory disorder, such as COPD or asthma.

In one embodiment, the nebulization composition is a solution comprising from about 0.00065% w/v to about 0.032% w/v of indacaterol or a salt thereof (e.g., a pharmaceutically acceptable salt thereof).

In another embodiment, the, the nebulization composition is a suspension comprising from about 0.00065% w/v to about 0.078% w/v of indacaterol or a salt thereof (e.g., a pharmaceutically acceptable salt thereof).

In one embodiment, the sterile pharmaceutical composition is a unit dose nebulizable pharmaceutical solution or suspension for inhalation comprising indacaterol or a salt thereof (e.g., a pharmaceutically acceptable salt thereof). The pharmaceutical solution or suspension may be administered in nebulized form to relieve bronchospasm in a subject, such as a subject suffering from COPD or asthma.

In one embodiment, any of the nebulization compositions described herein is substantially free (e.g., contains less than about 10% w/w, such as less than about 5% w/w, less than about 4% w/w, less than about 3% w/w, less than about 2% w/w, less than about 1% w/w, less than about 0.5% w/w, less than about 0.1% w/w, or less than about 0.01% w/w) of preservative, or is free of preservative.

In one embodiment, the present invention relates to a nebulization composition comprising

(i) indacaterol or a pharmaceutically acceptable salt thereof; and

(ii) water,

wherein the composition is free of any additional pharmaceutically acceptable excipients.

In a preferred embodiment, the indacaterol salt in any of the nebulization compositions described herein is indacaterol maleate, 5-[2-(5,6-Diethylindan-2-ylamino)-1-hydroxyethyl]-8-hydroxy-1H-quinolin-2-one maleate. In a more preferred embodiment, the indacaterol or pharmaceutically acceptable salt thereof (e.g., indacaterol maleate) in any of the nebulization compositions described herein is present as the (R)-isomer.

In an embodiment, the indacaterol or pharmaceutically acceptable salt thereof in any of the nebulization compositions described herein is micronized indacaterol maleate.

In another embodiment, the indacaterol or pharmaceutically acceptable salt thereof in any of the nebulization compositions described herein is sterile indacaterol maleate.

In yet another embodiment, the indacaterol or pharmaceutically acceptable salt thereof in any of the nebulization compositions described herein is sterile, micronized indacaterol maleate.

In yet another embodiment, any of the nebulization compositions described herein contains from about 5 μg/ml to about 1200 μg/ml of indacaterol maleate equivalent to indacaterol base, such as from about 5 μg/ml to about 20 μg/ml, from about 20 μg/ml to about 75 μg/ml, from about 75 μg/ml to about 150 μg/ml, from about 150 μg/ml to about 300 μg/ml, from about 300 μg/ml to about 600 μg/ml, from about 600 μg/ml to about 800 μg/ml, from about 800 μg/ml to about 1000 μg/ml, or from about 1000 μg/ml to about 1200 μg/ml of indacaterol maleate equivalent to indacaterol base.

One embodiment of the present invention is a nebulization composition, such as an aqueous solution or suspension, comprising indacaterol or a pharmaceutically acceptable salt thereof, such as indacaterol maleate, wherein the volume of the composition, such as an aqueous solution or suspension, is about 0.1 mL to about 6 mL, such as about 2 mL.

In yet another embodiment, the volume of any of the nebulization compositions described herein is about 0.1 ml to about 6 ml. Preferably, the volume of the nebulization composition is about 0.1 mL to about 6 mL, such as about 2 mL.

In one embodiment, any of the nebulization compositions described herein contains micronized indacaterol or a pharmaceutically acceptable salt thereof. In one embodiment, the indacaterol or pharmaceutically acceptable salt thereof has a mean particle size of about 0.1 micron to about 5 microns. In another embodiment, the indacaterol or pharmaceutically acceptable salt thereof has a D₉₀ of not more than 10 microns. In one embodiment, the indacaterol or pharmaceutically acceptable salt thereof has a mean particle size of about 0.1 micron to about 5 microns and a D₉₀ of not more than 10 microns. In yet another embodiment, any of the nebulization compositions described herein contains indacaterol or a pharmaceutically acceptable salt thereof in un-micronized form.

In one embodiment, the present invention relates to a nebulization composition comprising:

(i) indacaterol or a pharmaceutically acceptable salt thereof;

(ii) an isotonicity agent;

(iii) a buffer; and

(iv) optionally one or more pharmaceutically acceptable excipients selected from the group consisting of a complexing agent, a preservative, a surfactant, and any combination thereof.

In yet another embodiment, the present invention relates to nebulization composition comprising:

(i) indacaterol or a pharmaceutically acceptable salt thereof;

(ii) a pharmaceutically acceptable surfactant;

(iii) a pharmaceutically acceptable isotonicity adjusting agent;

(vi) a pH adjusting agents;

(v) optionally, a complexing agent; and

(vi) optionally, a pharmaceutically acceptable buffer;

wherein the pH of the nebulization composition is between about 2 and about 8, such as between about 2.0 and about 4.0.

In one embodiment, the nebulization composition comprises indacaterol or a pharmaceutically acceptable salt thereof, a pharmaceutically acceptable surfactant, an isotonicity agent, a buffer, a complexing agent and water for injection.

In another embodiment, the nebulization composition comprises indacaterol or a pharmaceutically acceptable salt thereof, polysorbate 80, sodium chloride, a buffer selected from sodium dihydrogen phosphate dihydrate, anhydrous disodium hydrogen phosphate or a combination thereof, edetate disodium, and water for injection.

In another embodiment, the nebulization composition comprises indacaterol or a pharmaceutically acceptable salt thereof, polysorbate 80, sodium chloride, a buffer selected from citric acid monohydrate, trisodium citrate dihydrate or a combination thereof, edetate disodium, and water for injection.

In another embodiment, the nebulization composition comprises indacaterol or a pharmaceutically acceptable salt thereof, a pharmaceutically acceptable surfactant, an isotonicity agent, a complexing agent, and water for injection.

In another embodiment, the nebulization composition comprises indacaterol or a pharmaceutically acceptable salt thereof, polysorbate 80, sodium chloride, edetate disodium, and water for injection.

In one embodiment, the nebulization composition comprises indacaterol or a pharmaceutically acceptable salt thereof, a pharmaceutically acceptable surfactant, an isotonicity agent, a buffer, and water for injection.

In another embodiment, the nebulization composition comprises indacaterol or a pharmaceutically acceptable salt thereof, polysorbate 80, sodium chloride, a buffer selected from sodium dihydrogen phosphate dihydrate, anhydrous disodium hydrogen phosphate or a combination thereof, and water for injection

In one embodiment, the nebulization composition comprises indacaterol or a pharmaceutically acceptable salt thereof, polysorbate 20, sodium chloride, a buffer selected from citric acid monohydrate, trisodium citrate dihydrate or a combination thereof, and water for injection

In one embodiment, the nebulization composition comprises indacaterol or a pharmaceutically acceptable salt thereof, a pharmaceutically acceptable surfactant, an isotonicity agent, and water for injection.

In another embodiment, the nebulization composition comprises indacaterol or a pharmaceutically acceptable salt thereof, polysorbate 80, sodium chloride, and water for injection.

In one embodiment, the nebulization composition comprises indacaterol or a pharmaceutically acceptable salt thereof, a surfactant selected from polyoxyethylene sorbitan monolaurate, sorbitan monolaurate or a combination thereof, sodium chloride and water for injection.

In another embodiment, the nebulization composition comprises indacaterol or a pharmaceutically acceptable salt thereof, polyoxyethylene sorbitan monolaurate, sodium chloride, and water for injection.

In another embodiment, the nebulization composition comprises indacaterol or a pharmaceutically acceptable salt thereof, sorbitan monolaurate, sodium chloride, and water for injection.

In one embodiment, the nebulization composition comprises indacaterol or a pharmaceutically acceptable salt thereof, an isotonicity agent, a buffer, a complexing agent, and water for injection.

In another embodiment, the nebulization composition comprises indacaterol or a pharmaceutically acceptable salt thereof, sodium chloride, a buffer selected from citric acid monohydrate, trisodium citrate dihydrate or a combination thereof, edetate disodium, and water for injection.

In one embodiment, the nebulization composition comprises indacaterol or a pharmaceutically acceptable salt thereof, an isotonicity agent, a buffer, and water for injection.

In another embodiment, the nebulization composition comprises indacaterol or a pharmaceutically acceptable salt thereof, sodium chloride, a buffer selected from citric acid monohydrate, trisodium citrate dihydrate or a combination thereof, and water for injection.

In one embodiment, the nebulization composition comprises indacaterol or a pharmaceutically acceptable salt thereof, an isotonicity agent, a buffering agent, optionally a complexing agent, optionally a preservative, and water for injection.

In an embodiment, the nebulization composition comprises indacaterol or a pharmaceutically acceptable salt thereof, sodium chloride, a buffer selected from tartaric acid, monosodium citrate or a combination thereof, edetate disodium, benzalkonium chloride, and water for injection.

In another embodiment, the nebulization composition comprises indacaterol or a pharmaceutically acceptable salt thereof, sodium chloride, a buffer selected from tartaric acid, monosodium citrate or a combination thereof, benzalkonium chloride, and water for injection.

In an embodiment, the nebulization composition comprises indacaterol or a pharmaceutically acceptable salt thereof, sodium chloride, a buffer selected from tartaric acid, monosodium citrate or a combination thereof, edetate disodium, and water for injection.

In one more embodiment, the nebulization composition comprises indacaterol or a pharmaceutically acceptable salt thereof, sodium chloride, a buffer selected from tartaric acid, monosodium citrate or a combination thereof, and water for injection.

In an embodiment, the nebulization composition comprises indacaterol or a pharmaceutically acceptable salt thereof, sodium chloride, a buffer selected from tartaric acid, sodium tartrate dihydrate or a combination thereof, edetate disodium, benzalkonium chloride, and water for injection.

In another embodiment, the nebulization composition comprises indacaterol or a pharmaceutically acceptable salt thereof, sodium chloride, a buffer selected from tartaric acid, sodium tartrate dihydrate or a combination thereof, benzalkonium chloride, and water for injection.

In an embodiment, the nebulization composition comprises indacaterol or a pharmaceutically acceptable salt thereof, sodium chloride, a buffer selected from tartaric acid, sodium tartrate dihydrate or a combination thereof, edetate disodium, and water for injection.

In one more embodiment, the nebulization composition comprises indacaterol or a pharmaceutically acceptable salt thereof, sodium chloride, a buffer selected from tartaric acid, sodium tartrate dihydrate or a combination thereof, and water for injection.

In an embodiment, the nebulization composition comprises indacaterol or a pharmaceutically acceptable salt thereof, sodium chloride, a buffer selected from phosphoric acid, sodium dihydrogen phosphate dihydrate or a combination thereof, edetate disodium, benzalkonium chloride, and water for injection.

In another embodiment, the nebulization composition comprises indacaterol or a pharmaceutically acceptable salt thereof, sodium chloride, a buffer selected from phosphoric acid, sodium dihydrogen phosphate dihydrate or a combination thereof, benzalkonium chloride, and water for injection.

In an embodiment, the nebulization composition comprises indacaterol or a pharmaceutically acceptable salt thereof, sodium chloride, a buffer selected from phosphoric acid, sodium dihydrogen phosphate dihydrate or a combination thereof, edetate disodium, and water for injection.

In one more embodiment, the nebulization composition comprises indacaterol or a pharmaceutically acceptable salt thereof, sodium chloride, a buffer selected from phosphoric acid, sodium dihydrogen phosphate dihydrate or a combination thereof, and water for injection.

In one embodiment, the nebulization composition comprises indacaterol or a pharmaceutically acceptable salt thereof, an isotonicity agent, a pH adjusting agent, optionally a complexing agent, optionally a preservative, and water for injection.

In an embodiment, the nebulization composition comprises indacaterol or a pharmaceutically acceptable salt thereof, sodium chloride, a pH adjusting agent, edetate disodium, benzalkonium chloride, and water for injection.

In another embodiment, the nebulization composition comprises indacaterol or a pharmaceutically acceptable salt thereof, sodium chloride, a pH adjusting agent, benzalkonium chloride, and water for injection.

In an embodiment, the nebulization composition comprises indacaterol or a pharmaceutically acceptable salt thereof, sodium chloride, a pH adjusting agent, edetate disodium, and water for injection.

In one more embodiment, the nebulization composition comprises indacaterol or a pharmaceutically acceptable salt thereof, sodium chloride, a pH adjusting agent, and water for injection.

In one embodiment, any of the nebulization compositions described herein, when administered by a nebulizer provides a mass median aerodynamic diameter (MMAD) of below about 10 microns, such as between about 4 and about 6 microns.

In another embodiment, any of the nebulization compositions described herein, when administered by a nebulizer provides a geometric standard deviation (GSD) of below about 5, such as between about 1 and about 3.

In another embodiment, any of the nebulization compositions described herein, when administered by a nebulizer device provides a fine particle dose which is not less than about 10%.

In another embodiment of any of the nebulization compositions described herein, the fine particle fraction (FPF) obtained following administration of the nebulizable composition in a nebulizer is about 10% to about 60%.

In another embodiment of any of the nebulization compositions described herein, the time taken to nebulize the nebulizable composition from the nebulizer device is about 1 to about 10 minutes.

In another embodiment of any of the nebulization compositions described herein, the nebulization composition exhibits a delivered dose between about 40% to about 80%.

In another embodiment of any of the nebulization compositions described herein, the nebulization composition is a stable composition. For example, the nebulization composition contains greater than about 80%, such as greater than about 85%, greater than about 90%, greater than about 95% or greater than about 98% of the initial amount of indacaterol or pharmaceutically acceptable salt thereof in the nebulization composition after being stored for 3 or 6 months or 1, 2 or 3 years at 25° C., when stored, e.g., in a suitable low density polyethylene (LDPE) container.

In another embodiment of any of the nebulization compositions described herein, the nebulization composition contains not more than about 0.1% of a monoethyl impurity of indacaterol (5-[(1R)-2-[(5-ethyl-2,3-dihydro-1H-inden-2-yl)amino]-1-hydroxyethyl]-8-hydroxy-2(1H)-quinolinone) or pharmaceutically acceptable salt thereof.

In another embodiment of any of the nebulization compositions described herein, the nebulization composition contains not more than about 0.05% of a benzyl impurity of indacaterol (5-[(1R)-2-[(5,6-diethyl-2,3-dihydro-1H-inden-2-yl)amino]-1-hydroxyethyl]-8-benzyloxy-1H-quinolin-2-one) or pharmaceutically acceptable salt thereof.

In another embodiment of any of the nebulization compositions described herein, the osmolality of the nebulization composition is between about 200 and about 500 mOsm/kg, such as between about 275 and about 325 mOsm/kg.

In another aspect, the present invention relates to a prepackaged, sterile, premixed, premeasured aqueous solution or suspension comprising indacaterol maleate. In one embodiment, the nebulization composition is a ready-to-use dosage form which does not require any mixing or dilution by the subject prior to administration. The nebulization composition may be administered for the relief of bronchospasm in a subject suffering from, for example, COPD or asthma.

In another aspect, the present invention relates to one or more prefilled containers containing a nebulization composition according to any of the embodiments described herein. In one embodiment, each container comprises a single unit dose of a nebulization composition according to any of the embodiments described herein, which may be used, e.g., for the treatment of COPD or asthma. In one embodiment, each container includes a sterile, premixed, premeasured, aqueous solution or suspension comprising a single unit dose of a therapeutically effective amount of indacaterol or its pharmaceutically acceptable salt thereof in a single container.

In another aspect, the present invention relates to one or more prefilled containers containing a nebulization composition according to any of the embodiments described herein, wherein the nebulization composition is in a solid dosage form, e.g. in a powder form, which can be reconstituted prior to nebulization with an appropriate diluent supplied in another prefilled container. The solid dosage form can be prepared by various methods such as, but not limited to, dry mixing, spray drying, and lyophilization.

In another aspect, the present invention relates to a method of administering indacaterol or a pharmaceutically acceptable salt thereof, comprising administering by inhalation to a subject in need thereof a nebulization composition according to any of the embodiments described herein.

In one embodiment, the method comprises administering by inhalation a nebulization composition according to any of the embodiments described herein at least once daily or twice daily.

In certain embodiments, the total daily dose of indacaterol or a pharmaceutically acceptable salt thereof (such as indacaterol maleate) administered in any of the methods described herein can range from about 10 to about 5000 μg/day, in a single or divided dose.

In another aspect, the present invention relates to a method of relieving bronchospasm (such as that associated with COPD or asthma) comprising administering by inhalation to a subject in need thereof a nebulization composition according to any of the embodiments described herein.

In another aspect, the present invention relates to a method of providing a faster onset of relief from bronchospasm (such as that associated with COPD or asthma) comprising administering by inhalation to a subject in need thereof a nebulization composition according to any of the embodiments described herein.

In another aspect, the present invention relates to a method of increasing the FEV1 values comprising administering by inhalation to a subject in need thereof a nebulization composition according to any of the embodiments described herein.

In another aspect, the present invention relates to a kit for administering a bronchodilator to relieve bronchospasm, for example, bronchospasm associated with COPD or asthma. The kit may comprise a nebulization composition according to any of the embodiments described herein.

In one embodiment, the kit comprises a nebulization composition according to any of the embodiments described herein in a prepackaged, premeasured, premixed and/or single unit dose form, e.g., for the treatment of COPD or asthma. In another embodiment, the prepackaged inhalation kit comprises one or more premixed, premeasured single unit dose vials comprising a nebulization composition according to any of the embodiments described herein, e.g., for the treatment of bronchospasm (such as that associated with COPD or asthma), and instructions for using the same.

Another embodiment is a kit comprising a nebulizer, instructions for using the nebulizer and a unit dose vial containing a nebulization composition according to any of the embodiments described herein.

In one embodiment, the time taken for administering a nebulization composition according to any of the embodiments described herein may be from about 1 minute to about 10 minutes.

In another aspect, the present invention relates to a kit for the treatment, prevention or amelioration or one or more symptoms of diseases or disorders associated with bronchoconstriction, comprising:

(i) a nebulizer; and

(ii) a nebulization composition for the treatment, prevention or amelioration or one or more symptoms of diseases or disorders associated with bronchoconstriction which comprises:

(a) indacaterol or a pharmaceutically acceptable salt thereof; and

(b) water.

In another aspect, the present invention relates to a kit for the treatment, prevention or amelioration or one or more symptoms of diseases or disorders associated with bronchoconstriction, comprising:

(i) a nebulizer; and

(ii) a nebulization composition according to any of the embodiments described herein for the treatment, prevention or amelioration or one or more symptoms of diseases or disorders associated with bronchoconstriction.

In another aspect, the present invention relates to a process for preparing a nebulization composition according to any of the embodiments described herein, the process comprising the steps of:

(i) dissolving an isotonicity agent, a buffer and a complexing agent in water for injection;

(ii) separately dispersing indacaterol or a pharmaceutically acceptable salt thereof in a surfactant in water for injection, and homogenizing the mixture;

(iii) sterilizing the solution or suspension of step (ii);

(iv) adding the solution or suspension of step (iii) to the product of step (i) and mixing for an adequate time;

(v) making up the weight of the solution or suspension of step (iv) using water for injection; and

(vi) filling the product of step (v) into LDPE vials

In another aspect, the present invention relates to a device comprising indacaterol or a pharmaceutically acceptable salt thereof (such as indacaterol maleate), for example, for use in relieving the symptoms of COPD or asthma.

In another aspect, the present invention relates to a method for improving user compliance and/or quality of life as compared to conventional treatments for COPD or asthma. In one embodiment, the method comprises initiating treatment with a nebulization composition according to any of the embodiments described herein, or a container, kit, or system according to any of the embodiments described herein.

The present invention provides convenient, fast and reliable treatment for COPD or asthma that represents an improvement over traditional treatments.

### DETAILED DESCRIPTION OF THE INVENTION

The indacaterol or pharmaceutically acceptable salt thereof present in any of the compositions described herein may be in any form such as in the form of an acid, salt, hydrate, polymorph, hemihydrate, or solvate, unless indicated otherwise. In one embodiment, the indacaterol or pharmaceutically acceptable salt thereof is indacaterol maleate. In one embodiment, the indacaterol or pharmaceutically acceptable salt thereof is anhydrous indacaterol maleate. In one embodiment, the indacaterol or pharmaceutically acceptable salt thereof is anhydrous amorphous indacaterol maleate.

In any of the compositions described herein, the indacaterol or pharmaceutically acceptable salt thereof may be provided in a variety of pharmaceutically acceptable vehicles, including, but not limited to, water or hydroalcoholic mixtures or other aqueous vehicles comprising a pharmaceutically acceptable amount of an osmotic agent.

As used herein, the term “effective amount” refers to an amount of an active agent, such as indacaterol or a pharmaceutically acceptable salt thereof (e.g., indacaterol maleate), effective to treat, reduce, alleviate, ameliorate, eliminate or prevent one or more symptoms of a condition sought to be treated, or alternately, the condition sought to be avoided, or to otherwise produce a clinically recognizable favorable change in the condition or its effects.

The nebulization compositions described herein may contain indacaterol maleate in micronized form. Suitable micronization techniques that can be employed include, for example, microfluidizer, high pressure homogenizer, ball mill, sonication and other techniques commonly known in the art.

The nebulization compositions according to any of the embodiments described herein may have a pH of between about 2 and about 8, such as between about 2.0 and about 4.0. The pH may be adjusted by the addition of one or more pharmaceutically acceptable acids. Examples of suitable pharmaceutically acceptable acids include, but are not limited to, inorganic acids, such as hydrochloric acid, hydrobromic acid, nitric acid, sulfuric acid, and phosphoric acid, and any combination thereof. Other examples of other suitable pharmacologically acceptable acids include, but are not limited to, organic acids, such as ascorbic acid, citric acid, malic acid, maleic acid, tartaric acid, succinic acid, fumaric acid, acetic acid, formic acid, and/or propionic acid. In one embodiment, the pH is adjusted with 1N hydrochloric acid or 1N sulfuric acid. In another embodiment, the pH is adjusted with one or more organic acids selected from ascorbic acid, fumaric acid, citric acid and any combination thereof. A preferred organic acid is citric acid. If desired, mixtures of the abovementioned acids may also be used, particularly in the case of acids which have other properties in addition to their acidifying properties, e.g., those which act as flavorings or antioxidants, such as for example citric acid or ascorbic acid.

Any of the nebulization compositions described herein may optionally include a buffer. Suitable general and biological buffers that may be used, such as those in the pH range of about 2 to about 8 include, but are not limited to, acetate, barbital, borate, Britton-Robinson, cacodylate, citrate, collidine, formate, maleate, McIlvaine, phosphate, Prideaux-Ward, phosphate, citrate, borate, succinate, citrate-phosphate-borate (Teorell-Stanhagen), veronal acetate, 2-(N-morpholino)ethanesulfonic acid (MES), BIS-TRIS, N-(2-Acetamido)iminodiacetic acid, N-(2-Acetamido)-2-aminoethanesulfonic acid (ADA), piperazine-N,N′-bis(2-ethanesulfonic acid) (PIPES), β-Hydroxy-4-morpholinepropanesulfonic acid (MOPSO), 1,3-bis(tris(hydroxymethyl)methylamino)propane (BIS TRIS Propane), N,N-Bis(2-hydroxyethyl)-2-aminoethanesulfonic acid (BES), (3-(N-morpholino)propanesulfonic acid) (MOPS), N-[Tris(hydroxymethyl)methyl]-2-aminoethanesulfonic acid (TES), (4-(2-hydroxyethyl)-1-piperazineethanesulfonic acid) (HEPES), N,N-Bis(2-hydroxyethyl)-3-amino-2-hydroxypropanesulfonic acid (DIPSO), (3-(N-morpholino)propanesulfonic acid) (MOBS), 2-Hydroxy-3-[tris(hydroxymethyl)methylamino]-1-propanesulfonic acid (TAPSO), [Tris(hydroxymethyl)aminomethane] (TRIZMA), (2-Hydroxyethyl)-piperazine-N-2-hydroxypropanesulfonic acid) (HEPPSO), Piperazine-1,4-bis(2-hydroxypropanesulfonic acid) dihydrate (POPSO), Triethanolamine (TEA), 4-(2-Hydroxyethyl)-1-piperazinepropanesulfonic acid (EPPS), (N-Tris(Hydroxymethyl) Methylglycine) (TRICINE), Diglycine (GLY-GLY), N,N-Bis(2-hydroxyethyl)glycine (BICINE), N-(2-Hydroxyethyl)piperazine-N′-(4-butanesulfonic acid) (HEPBS), N-[Tris(hydroxymethyl)methyl]-3-aminopropanesulfonic acid (TAPS), and 2-Amino-2-methyl-1,3-propanediol (AMPD) buffers. In certain embodiments, the buffer is sodium hydrogen phosphate dihydrate, anhydrous disodium hydrogen phosphate buffer, citric acid monohydrate, trisodium citrate dihydrate, or any combination thereof.

Any of the nebulization compositions described herein may contain from about 0.5% to about 1.5% w/v of sodium dihydrogen phosphate dihydrate, or from about 0.05% to about 0.5% w/v of anhydrous disodium hydrogen phosphate, or from about 0.05% to about 2.0% % w/v of citric acid anhydrous/monohydrate, or from about 0.1% to about 1.83% w/v of sodium citrate dihydrate/monosodium citrate or from about 0.05% to about 1.0% tartaric acid/phosphoric acid or from about 0.5% to about 1.5% sodium tartrate dihydrate, or any combination of any of the foregoing.

The osmolality of any of the nebulization compositions described herein may be from about 200 to about 500 mOsm/kg, such as from about 275 to about 325 mOsm/kg. Any of the nebulization compositions described herein may comprise from about 0.4 to about 1.0 weight percent of an ionic salt.

Suitable tonicity adjusting agents for use in any of the nebulization compositions described herein include, but are not limited to, ammonium carbonate, ammonium chloride, ammonium lactate, ammonium nitrate, ammonium phosphate, ammonium sulfate, ascorbic acid, bismuth sodium tartrate, boric acid, calcium chloride, calcium disodium edetate, calcium gluconate, calcium lactate, citric acid, dextrose, diethanolamine, dimethyl sulfoxide, edetate disodium, edetate trisodium, fluorescein sodium, fructose, galactose, glycerin, lactic acid, lactose, magnesium chloride, magnesium sulfate, mannitol, polyethylene glycol, potassium acetate, potassium chlorate, potassium chloride, potassium iodide, potassium nitrate, potassium phosphate, potassium sulfate, propylene glycol, silver nitrate, sodium acetate, sodium bicarbonate, sodium biphosphate, sodium bisulfite, sodium borate, sodium bromide, sodium cacodylate, sodium carbonate, sodium chloride, sodium citrate, sodium iodide, sodium lactate, sodium metabisulfite, sodium nitrate, sodium nitrite, sodium phosphate, sodium propionate, sodium succinate, sodium sulfate, sodium sulfite, sodium tartrate, sodium thiosulfate, sorbitol, sucrose, tartaric acid, triethanolamine, urea, urethan, uridine, zinc sulfate, and any combination of any of the foregoing.

Any of the nebulization compositions described herein may contain between about 0.25% and about 1.13% w/v, such as about 0.9% w/v of sodium chloride.

Suitable osmotic adjusting agents for use in any of the nebulization compositions described herein include, but are not limited to, sodium chloride, potassium chloride, zinc chloride, calcium chloride and any combination of any of the foregoing. Other osmotic adjusting agents for use in any of the nebulization compositions described herein include, but are not limited to, mannitol, glycerol, dextrose, and any combination of any of the foregoing.

Any cosolvent that is suitable for inhalation and capable of dissolving or dispersing indacaterol or a pharmaceutically acceptable salt thereof in a mixture of cosolvent and water can be used in the nebulization compositions described herein. Examples of suitable cosolvents include, for example, alcohols, ethers, hydrocarbons, perfluorocarbons, and any combination thereof. In one embodiment, the cosolvent is a short chain polar alcohol. For example, the cosolvent is an aliphatic alcohol having from one to six carbon atoms, such as ethanol or isopropanol. In one embodiment the cosolvent is ethanol. Examples of suitable hydrocarbons include, but are not limited to, n-butane, isobutane, pentane, neopentane and isopentanes. Examples of suitable ethers include, but are not limited to, dimethyl ether and diethyl ether. Examples of suitable perfluorocarbons include, but are not limited to, perfluoropropane, perfluorobutane, perfluorocyclobutane, and perfluoropentane.

Suitable nonionic surfactants for use in any of the nebulization compositions described herein include, but are not limited to, all substances of this type that can normally be used in compositions. For example, suitable nonionic surfactants include polyethylene glycol ethers of linear alcohols, reaction products of fatty acids with ethylene oxide and/or propylene oxide, polyvinyl alcohol, polyvinylpyrrolidone, copolymers of polyvinyl alcohol and polyvinylpyrrolidone, polysorbates, polyoxyethylene sorbitan monolaurate, sorbitan monolaurate, polyethylene oxide-polypropylene oxide block copolymers and also copolymers of (meth)acrylic acid and (meth)acrylic esters, and also alkyl ethoxylates and alkylaryl ethoxylates, which optionally may be phosphated and optionally may be neutralized with bases, such as sorbitol ethoxylates, and any combination of any of the foregoing.

Suitable anionic surfactants for use in any of the nebulization compositions described herein include, but are not limited to, all substances of this type that can normally be used in compositions. For example, suitable anionic surfactants include alkali metal salts and alkaline earth metal salts of alkylsulphonic acids or alkylarylsulphonic acids, and any combination of any of the foregoing.

Examples of suitable cationic surfactants for use in any of the nebulization compositions described herein include, but are not limited to, ammnonium bromides.

Examples of suitable nonionic surfactants for use in any of the nebulization compositions described herein include, but are not limited to alkyl ethoxylates, alkyl gluccosides and alkyl phenol ethoxylates, and any combination of any of the foregoing.

Examples of suitable amphoteric surfactants for use in any of the nebulization compositions described herein include, but are not limited to, betaines, alkyl betaines, alkyl amido betaines, alkyl amphoacetates, and amphodiacetates, and any combination of any of the foregoing.

A preferred anionic surfactant is sodium dodecyl sulfate (SDS). A preferred cationic surfactant is dodecyl trimethyl ammonium bromide.

=In any of the nebulization compositions described herein, the surfactant comprises polysorbates, sorbitans or a combination of any of the foregoing.

In certain embodiments of any of the nebulization compositions described herein, the nebulization composition comprises from about 0.01% to about 0.40% w/v polysorbate 80/polysorbate 20, or from about 0.0001% to 0.4% w/v of polyoxyethylene sorbitan monolaurate, or from about 0.01% to 0.4% w/v of sorbitan monolaurate.

Suitable antioxidants for use in any of the nebulization compositions described herein include, but are not limited to, ascorbic acid, for example, provided that it has not already been used to adjust the pH, vitamin A, vitamin E, tocopherols and similar vitamins or pro-vitamins occurring in the human body, and any combination of any of the foregoing.

Any of the nebulization compositions described herein may also contain a complexing agent. Suitable examples include, but are not limited to, EDTA and salts thereof, such as edetate disodium. In one embodiment, the nebulization compositions described herein comprise between about 0.0001% and about 0.75% w/v edetate disodium, such as between about 0.01% and about 0.05% w/v edetate disodium.

Any of the nebulization compositions described herein may be contained in a unit-dose, low-density polyethylene (LDPE) container, polypropylene container, or a cyclic polyolefin container. Each unit-dose container may be disposed in a foil pouch, and each foil pouch may contain 2 or more unit-dose containers. Each foil pouch containing the unit dose container may be disposed in a shelf carton.

The kits described herein may provide such containers in prepackaged form. In one embodiment, a container with a TWIST-FLEX™ top is preferred, such top comprising an easy-to-grip tab-like handle such that the container may be opened, for example, by twisting off the tab by hand. The TWIST-FLEX™ top is advantageous in that it allows for easy dispensing of the nebulization composition, prevents spillage and eliminates the need to open the container or tearing by cutting or tearing off the top, or the like, thereby reducing cross-contamination. One or more of the semi-permeable single unit dose containers may be prepackaged in aluminum foil pouch, such that the foil provides a protective barrier against environmental contaminants and light as it helps to improves the shelf-life and stability of the nebulization composition. Dispensing vials may include, but are not limited to, any container comprising glass, low density polyethylene, polypropylene, cyclic polyolefins or any other material capable of preventing the nebulization composition from leaking out of the container. The vial may be enclosed by any conventional means including, but not limited to, screw cap, heat seal, snap-on top, flip-top, twist-off stopper, and peel away top.

One or more prefilled containers containing a nebulization composition according to any of the embodiments described herein can also be provided. Each container comprises a single unit dose of a nebulization composition according to any of the embodiments described herein for the treatment of, for example, COPD or asthma. Each such container includes a sterile, premixed, premeasured, aqueous solution or suspension comprising a single unit dose of a therapeutically effective amount of indacaterol or a pharmaceutically acceptable salt thereof in a single container. In one embodiment, the present invention related to a prefilled container containing about 2 mL of a nebulization composition according to any of the embodiments described herein comprising about 5 μg to about 1200 μg of indacaterol maleate equivalent to indacaterol base. The nebulization composition may additionally contain a surfactant, a buffer, an isotonicity agent and optionally a complexing agent.

The nebulization compositions described herein may be administered by a suitable nebulizer. Suitable nebulizers include, but are not limited to, a jet nebulizer, an ultrasonic nebulizer, vibrating mesh nebulizer and a breath actuated nebulizer. Preferably, the nebulizer is a jet nebulizer connected to an air compressor with adequate airflow. The nebulizer being equipped with a mouthpiece or suitable face mask. Exemplary jet nebulizers for use herein include, but are not limited to, Pari LCplus/ProNeb, Pari LC plus/ProNeb Turbo, Pari LC plus/Dura Neb 1000 & 2000, Pari LC plus/Walkhaler, Pari LC plus/Pari Master, Pari LC star, Omron CompAir XL Portable Nebulizer System (NE-C 18 and JetAir Disposable nebulizer), Omron CompAir Elite Compressor Nebulizer System (NE-C21 and Elite Air Reusable Nebulizer), Pari LC Plus or Pari LC Star nebulizer with Proneb Ultra compressor, Pulmo-aide, Pulmo-aide LT, Pulmo-aide traveler, Invacare Passport, Inspiration Healthdyne 626, Pulmo-Neb Traverler, DeVilbiss 646, Whisper Jet, Acorn II, Misty-Neb, Allied aerosol, Schuco Home Care, Lexan Plasic Pocet Neb, SideStream Hand Held Neb, Mobil Mist, Up-Draft, Up-Draft II, T Up-Draft, ISO-NEB, AVA-NEB, Micro Mist, and PulmoMate. Exemplary ultrasonic nebulizers for use herein include MicroAir, UltraAir, Siemens Ultra Nebulizer 145, CompAir, Pulmosonic, Scout, 5003 Ultrasonic Neb, 5110 Ultrasonic Neb, 5004 Desk Ultrasonic Nebulizer, Mystique Ultrasonic, Luminscope's Ultrasonic Nebulizer, Medisana Ultrasonic Nebulizer, Microstat Ultrasonic Nebulizer, and MABISMist Hand Held Ultrasonic Nebulizer. Other nebulizers for use herein include 5000 Electromagnetic Neb, 5001 Electromagnetic Neb 5002 Rotary Piston Neb, Lumineb I Piston Nebulizer 5500, AERONEB™ Portable Nebulizer System, AERODOSE™ Inhaler, AeroEclipse Breath Actuated Nebulizer, HALOLITE™ system (Profile Therapeutics), AKITA® systems (InaMed, Germany), Mystic system (BattellePharma), RESPIMAT® (Boehringer Ingelheim), Microbase®, AERX® (Aradigm), and E-FLOW™ (Pari). Additionally, the compositions described herein can also be nebulized using inhalers other than those described above, for example jet-stream inhalers or by breath actuated jet nebulizers.

The nebulization compositions described herein demonstrate a fine particle dose of about 10% to about 80% and a fine particle fraction (FPF) which is about 10% to about 80%. A geometric standard deviation (GSD) of about 0.5 to 5 may be observed when the nebulization compositions described herein are administered through a nebulizer. The nebulization compositions described herein demonstrate a mass mean aerodynamic diameter (MMAD) of less than about 10 microns when administered through a nebulizer device. The respirable dose delivery rate for the nebulization composition described herein is about 10% to 80%.

### EXAMPLES

### Example 1

| | Amount in % w/v | | | |
|---|---|---|---|---|
| # | A | B | C | D |
| Indacaterol maleate | 0.0013 to 0.019 | | | |
| Sodium chloride | 0.800 | 0.800 | 0.800 | 0.800 |
| Tartaric Acid | 0.250 | 0.250 | 0.250 | 0.250 |
| Monosodium citrate | 0.100 | 0.100 | 0.100 | 0.100 |
| Edetate disodium | 0.010 | — | 0.010 | — |
| Benzalkonium chloride | 0.020 | 0.020 | — | — |
| Water for injection | q.s 2 ml | q.s 2 ml | q.s 2 ml | q.s 2 ml |

Manufacturing Process
1. Dissolve sodium chloride, edetate disodium, tartaric acid and monosodium citrate in water for injection with stirring. Check the clarity of the solution.
2. Add indacaterol maleate to the product of step 1.
3. Stir/homogenize/sonicate until a clear solution is obtained.
4. Add benzalkonium chloride (BKC) to the product of step 3 and mix. Make up the volume with water for injection.
5. Filter the product of step 4 through a sterilizing grade filter.
6. Fill the product into LDPE smartules/HDPE, PP, LDPE bottles/amber glass bottles/amber glass vials in single dose or multidose containers.

### Example 2

| | Amount in % w/v | | | |
|---|---|---|---|---|
| # | A | B | C | D |
| Indacaterol maleate | 0.0013 to 0.019 | | | |
| Sodium chloride | 0.800 | 0.800 | 0.800 | 0.800 |
| Tartaric acid | 0.250 | 0.250 | 0.250 | 0.250 |
| Sodium tartrate | | | | |
| dihydrate | 0.100 | 0.100 | 0.100 | 0.100 |
| Edetate disodium | 0.010 | — | 0.010 | — |
| Benzalkonium chloride | 0.020 | 0.020 | — | — |
| Water for injection | q.s 2 ml | q.s 2 ml | q.s 2 ml | q.s 2 ml |

Manufacturing Process
1. Dissolve sodium chloride, edetate disodium, tartaric acid and sodium tartrate dihydrate in water for injection under stirring. Check clarity of the solution.
2. Add indacaterol maleate to the product of step 1.
3. Stir/homogenize/sonicate until a clear solution is obtained.
4. Add benzalkonium chloride (BKC) to the product of step 3 and mix. Make up the volume with water for injection.
5. Filter the product of step 4 through a sterilizing grade filter.
6. Fill the product into LDPE smartules/HDPE, PP, LDPE bottles/amber glass bottles/amber glass vials in single dose or multidose containers.

### Example 3

| | Amount in % w/v | | | |
|---|---|---|---|---|
| # | A | B | C | D |
| Indacaterol maleate | 0.0013 to 0.019 | | | |
| Sodium chloride | 0.800 | 0.800 | 0.800 | 0.800 |
| Phosphoric acid | 0.034 | 0.034 | 0.034 | 0.034 |
| Sodium dihydrogen | | | | |
| phosphate dihydrate | 0.078 | 0.078 | 0.078 | 0.078 |
| Edetate disodium | 0.010 | — | 0.010 | — |
| Benzalkonium chloride | 0.020 | 0.020 | — | — |
| Water for injection | q.s 2 ml | q.s 2 ml | q.s 2 ml | q.s 2 ml |

Manufacturing Process
1. Dissolve sodium chloride, edetate disodium, phosphoric acid, sodium dihydrogen phosphate dihydrate and benzalkonium chloride in water for injection with stirring. Check the clarity of the solution.
2. Add indacaterol maleate to the product of step 1.
3. Stir/homogenize/sonicate until a clear solution is obtained.
4. Add benzalkonium chloride (BKC) to the product of step 3 and mix. Make up the volume with water for injection.
5. Filter the product of step 4 through a sterilizing grade filter.
6. Fill the product into LDPE smartules/HDPE, PP, LDPE bottles/amber glass bottles/amber glass vials in single dose or multidose containers.

### Example 4

| | Amount in % w/v | | | |
|---|---|---|---|---|
| # | A | B | C | D |
| Indacaterol maleate | 0.0013 to 0.019 | | | |
| Sodium chloride | 0.900 | 0.900 | 0.900 | 0.900 |
| Edetate disodium | 0.010 | 0.010 | — | — |
| Benzalkonium Chloride | 0.020 | — | 0.020 | — |
| HCl | q.s pH | q.s pH | q.s pH | q.s pH |
| Water for injection | q.s 2 ml | q.s 2 ml | q.s 2 ml | q.s 2 ml |

Manufacturing Process
1. Dissolve sodium chloride in water for injection with stirring. Check the clarity of the solution. Adjust the pH of the solution to pH 2.7 with hydrochloric acid.
2. Add indacaterol maleate to the product of step 1.
3. Stir/homogenize/sonicate until a clear solution is obtained.
4. Add benzalkonium chloride (BKC) to the product of step 3 and mix. Make up the volume with water for injection.
5. Filter the product of step 4 through a sterilizing grade filter.
6. Fill the product into LDPE smartules/HDPE, PP, LDPE bottles/amber glass bottles/amber glass vials in single dose or multidose containers.

### Example 5

### Indacaterol Suspension for Nebulization

| | Amount in % w/v | | | | | | |
|---|---|---|---|---|---|---|---|
| Ingredients | A | B | C | D | E | F | G |
| Indacaterol | 0.00065-0.078 | | | | | | |
| maleate | | | | | | | |
| Polysorbate | 0.02 | 0.02 | 0.02 | 0.02 | — | — | — |
| 80 | | | | | | | |
| Polyoxyethylene | — | — | — | — | 0.1 | 0.1 | — |
| sorbitan | | | | | | | |
| monolaurate | | | | | | | |
| Sorbitan | — | — | — | — | 0.02 | — | 0.02 |
| monolaurate | | | | | | | |
| Sodium | 0.9 | 0.9 | 0.9 | 0.9 | 0.9 | 0.9 | 0.9 |
| chloride | | | | | | | |
| Sodium | 0.94 | — | 0.94 | — | — | — | — |
| dihydrogen | | | | | | | |
| phosphate | | | | | | | |
| dihydrate | | | | | | | |
| Anhydrous | 0.175 | — | 0.175 | — | — | — | — |
| disodium | | | | | | | |
| hydrogen | | | | | | | |
| phosphate | | | | | | | |
| Edetate | 0.02 | 0.02 | — | — | — | — | — |
| disodium | | | | | | | |
| Water for | q.s | q.s | q.s | q.s | q.s | q.s | q.s |
| injection | | | | | | | |

Manufacturing Process
1. Dissolve sodium chloride, edetate disodium, sodium dihydrogen phosphate dihydrate, anhydrous disodium hydrogen phosphate, polyoxyethylene sorbitan monolaurate and sorbitan monolaurate in water for injection with stirring. Check the clarity of the solution.
2. Mix indacaterol maleate, polysorbate 80 and water for injection in separate vessel, homogenize for 15 minutes and subject to sterilization by autoclave.
3. Add the product of step 2 to the product of step 1 with stirring and mix for 15 minutes. Make up the volume with water for injection.
4. Fill the bulk product into LDPE vials.

### Example 6

### Indacaterol Solution for Nebulization

| | Amount in % w/v | | | | | |
|---|---|---|---|---|---|---|
| Ingredients | A | B | C | D | E | F |
| Indacaterol | 0.00065-0.032 | | | | | |
| maleate | | | | | | |
| Sodium | 0.9 | 0.9 | 0.9 | 0.9 | 0.9 | 0.9 |
| chloride | | | | | | |
| Citric acid | 1.923 | 1.923 | 0.961 | 3.845 | 3.845 | 3.845 |
| monohydrate | | | | | | |
| Trisodium | 0.25 | 0.25 | 0.125 | 0.5 | 0.5 | 0.5 |
| citrate | | | | | | |
| dihydrate | | | | | | |
| Edetate | 0.01-0.02 | — | — | — | — | 0.02 |
| disodium | | | | | | |
| Polysorbate | — | — | — | — | 0.02 | — |
| 20 | | | | | | |
| Polysorbate | — | — | — | — | — | 0.02 |
| 80 | | | | | | |
| Water for | q.s | q.s | q.s | q.s | q.s | q.s |
| injection | | | | | | |

Manufacturing Process:
1. Dissolve sodium chloride, edetate disodium, citric acid monohydrate and trisodium citrate dihydrate in water for injection with stirring. Check the clarity of the solution.
2. Add indacaterol maleate to a mix polysorbate 20/polysorbate 80 and add the resulting product to the product of step 1 with stirring.
3. Sonicate the result mixture until a clear solution is obtained. Make up the volume with water for injection.
4. Filter the product of step 3 solution through sterilizing grade filter.
5. Fill the resulting bulk product into LDPE vials.

### Examples 7-12

| Example No. | 7 | 8 | 9 | 10 | 11 | 12 |
|---|---|---|---|---|---|---|
| Indacaterol maleate | 100 | 100 | 100 | 100 | 100 | 150 |
| (Conc. in μg/2 ml) | | | | | | |
| Citric acid monohydrate | 1.98 | 1.95 | 1.92 | 1.87 | 1.85 | 1.85 |
| (% w/v) | | | | | | |
| Trisodium citrate | 0.18 | 0.21 | 0.25 | 0.32 | 0.25 | 0.25 |
| (% w/v) | | | | | | |
| Sodium chloride (% w/v) | 0.9 | 0.9 | 0.9 | 0.9 | 0.9 | 0.6 |
| Edetate disodium | — | — | — | — | 0.01 | — |
| (% w/v) | | | | | | |
| Water | q.s | q.s | q.s | q.s | q.s | q.s |
| pH | 2.5 | 2.6 | 2.7 | 2.8 | 2.7 | 2.7 |

Manufacturing Process
1. Dissolve sodium chloride, edetate disodium (optional), citric acid monohydrate and trisodium citrate in water for injection with stirring. Check the clarity of the solution.
2. Add indacaterol maleate to the product of step 1 with stirring.
3. Sonicate the resulting mixture until a clear solution is obtained. Make up the volume with water for injection.
4. Filter the product of step 3 solution through a sterilizing grade filter.
5. Fill the resulting bulk product into LDPE vials.

The tables below present stability data for the nebulization compositions described in Examples 7-12.

### A. Indacaterol 100 μg/2 ml Composition with Buffer pH 2.5 (Example 7)

| | Pack | | | | | | | | | | | |
|---|---|---|---|---|---|---|---|---|---|---|---|---|
| | LDPE Respules | | | | | | Amber Glass Vials | | | | | |
| | Test | | | | | | | | | | | |
| | | | | Stage | | | | | | Stage | | |
| | | | | III | | | | | | III | | |
| | | | Monoethyl | (Benzyl | Single | Total | | | Monoethyl | (Benzyl | Single | Total |
| | Assay | pH | Impurity | Indacaterol) | Max | Impurity | Assay | pH | Impurity | Indacaterol) | Max | Impurity |
| Initial | 98.7 | 2.48 | 0.05 | 0.01 | 0.07 | 0.48 | 98.7 | 2.48 | 0.05 | 0.01 | 0.07 | 0.48 |

Stability condition: 40 ± 2° C./75 ± 5% RH

| | | | | | | | | | | | | |
|---|---|---|---|---|---|---|---|---|---|---|---|---|
| 1 M | 100.0 | 2.47 | 0.05 | ND | 0.06 | 0.48 | 98.5 | 2.46 | 0.05 | 0.02 | 0.20 | 0.93 |
| 2 M | 99.8 | 2.46 | 0.04 | ND | 0.06 | 0.42 | 95.5 | 2.45 | 0.05 | 0.02 | 0.59 | 1.81 |
| 3 M | 96.7 | 2.45 | 0.04 | 0.02 | 0.66 | 1.57 | 95.7 | 2.43 | 0.06 | 0.02 | 0.75 | 2.30 |

| | | | | | | | |
|---|---|---|---|---|---|---|---|
| 6 M | NA | 89.3 | 2.46 | 0.06 | 0.03 | 1.63 | 4.16 |

Stability condition: 5 ± 3° C.

| | | | | | | | | | | | | |
|---|---|---|---|---|---|---|---|---|---|---|---|---|
| 1 M | 19.5 | 2.46 | 0.04 | ND | 0.09 | 0.27 | 98.4 | 2.47 | 0.05 | 0.01 | 0.08 | 0.51 |
| 2 M | 24.0 | 2.45 | 0.04 | ND | 0.05 | 0.21 | 98.7 | 2.46 | 0.05 | 0.01 | 0.07 | 0.55 |
| 3 M | 19.3 | 2.43 | 0.03 | ND | 0.02 | 0.14 | 96.2 | 2.44 | 0.05 | 0.01 | 0.08 | 0.64 |
| 6 M | | | | | | | 97.7 | 2.46 | 0.05 | 0.01 | 0.10 | 0.82 |

Stability condition: 25 ± 2° C./60 ± 5% RH

| | | | | | | | | | | | | |
|---|---|---|---|---|---|---|---|---|---|---|---|---|
| 1 M | 99.2 | 2.48 | 0.05 | ND | 0.08 | 0.50 | 99.8 | 2.47 | 0.05 | 0.01 | 0.09 | 0.57 |
| 2 M | 98.2 | 2.47 | 0.05 | ND | 0.06 | 0.47 | 99.0 | 2.47 | 0.05 | 0.01 | 0.12 | 0.72 |
| 3 M | 97.9 | 2.45 | NA | NA | NA | NA | 93.8 | 2.46 | NA | NA | NA | NA |

| | | |
|---|---|---|
| 6 M | NA | NA |

Stability condition: 30 ± 2° C./75 ± 5% RH

| | | | | | | | | | | | | |
|---|---|---|---|---|---|---|---|---|---|---|---|---|
| 1 M | 100.0 | 2.47 | 0.05 | ND | 0.06 | 0.45 | 99.4 | 2.46 | 0.05 | 0.01 | 0.10 | 0.69 |
| 2 M | NA | NA | NA | NA | NA | NA | NA | NA | NA | NA | NA | NA |
| 3 M | 97.1 | 2.46 | 0.05 | ND | 0.06 | 0.38 | 97.1 | 2.47 | 0.05 | 0.02 | 0.37 | 1.29 |

| | | | | | | | |
|---|---|---|---|---|---|---|---|
| 6 M | NA | 94.4 | 2.46 | 0.05 | 0.03 | 0.48 | 1.67 |

### B. Indacaterol 100 μg/2 ml Composition with Buffer pH 2.6 (Example 8)

| | Pack | | | | | | | | | | | |
|---|---|---|---|---|---|---|---|---|---|---|---|---|
| | LDPE Respules | | | | | | Amber Glass Vials | | | | | |
| | Test | | | | | | | | | | | |
| | | | | Stage | | | | | | Stage | | |
| | | | | III | | | | | | III | | |
| | | | Monoethyl | (Benzyl | Single | Total | | | Monoethyl | (Benzyl | Single | Total |
| | Assay | pH | Impurity | Indacaterol) | Max | Impurity | Assay | pH | Impurity | Indacaterol) | Max | Impurity |
| Initial | 97.0 | 2.58 | 0.07 | 0.01 | 0.09 | 0.63 | 97.0 | 2.58 | 0.07 | 0.01 | 0.09 | 0.63 |

Stability condition: 40 ± 2° C./75 ± 5% RH

| | | | | | | | | | | | | |
|---|---|---|---|---|---|---|---|---|---|---|---|---|
| 1 M | 101.1 | 2.57 | 0.07 | ND | 0.09 | 0.69 | 98.9 | 2.57 | 0.07 | ND | 0.21 | 1.07 |
| 2 M | 95.3 | 2.56 | 0.06 | ND | 0.09 | 0.58 | 97.1 | 2.56 | 0.07 | 0.01 | 0.26 | 1.34 |
| 3 M | 97.5 | 2.54 | 0.06 | 0.01 | 0.09 | 0.63 | 96.5 | 2.55 | 0.08 | 0.02 | 0.97 | 2.76 |

| | | | | | | | |
|---|---|---|---|---|---|---|---|
| 6 M | NA | 80.6 | 2.56 | 0.09 | 0.04 | 2.49 | 5.85 |

Stability condition: 5 ± 3° C.

| | | | | | | | | | | | | |
|---|---|---|---|---|---|---|---|---|---|---|---|---|
| 1 M | 25.7 | 2.56 | 0.04 | ND | 0.05 | 0.38 | 97.4 | 2.56 | 0.06 | 0.01 | 0.09 | 0.67 |
| 2 M | 24.2 | 2.55 | 0.04 | ND | 0.06 | 0.24 | 98.6 | 2.55 | 0.06 | ND | 0.08 | 0.64 |
| 3 M | 19.5 | 2.53 | 0.04 | ND | 0.05 | 0.22 | 96.5 | 2.53 | 0.06 | ND | 0.09 | 0.77 |

| | | | | | | | |
|---|---|---|---|---|---|---|---|
| 6 M | NA | 95.4 | 2.57 | 0.06 | ND | 0.13 | 0.87 |

Stability condition: 25 ± 2° C./60 ± 5% RH

| | | | | | | | | | | | | |
|---|---|---|---|---|---|---|---|---|---|---|---|---|
| 1 M | 99.3 | 2.57 | 0.07 | ND | 0.09 | 0.66 | 98.9 | 2.57 | 0.06 | ND | 0.10 | 0.72 |
| 2 M | 98.9 | 2.57 | 0.06 | ND | 0.08 | 0.59 | 96.7 | 2.56 | 0.06 | 0.01 | 0.14 | 0.85 |
| 3 M | NA | NA | NA | NA | NA | NA | 96.3 | 2.55 | NA | NA | NA | NA |

| | | |
|---|---|---|
| 6 M | NA | NA |

Stability condition: 30 ± 2° C./75 ± 5% RH

| | | | | | | | | | | | | |
|---|---|---|---|---|---|---|---|---|---|---|---|---|
| 1 M | 100.0 | 2.56 | 0.07 | ND | 0.09 | 0.67 | 98.6 | 2.56 | 0.06 | 0.01 | 0.12 | 0.86 |
| 2 M | NA | NA | NA | NA | NA | NA | NA | NA | NA | NA | NA | NA |
| 3 M | 96.5 | 2.54 | 0.07 | ND | 0.09 | 0.61 | 97.1 | 2.55 | 0.07 | 0.01 | 0.29 | 1.24 |

| | | | | | | | |
|---|---|---|---|---|---|---|---|
| 6 M | NA | 93.9 | 2.58 | 0.07 | 0.02 | 0.46 | 1.66 |

### C. Indacaterol 100 μg/2 ml Composition with Buffer pH 2.7 (Example 9)

| | Pack | | | | | | | | | | | |
|---|---|---|---|---|---|---|---|---|---|---|---|---|
| | LDPE Respules | | | | | | Amber Glass Vials | | | | | |
| | Test | | | | | | | | | | | |
| | | | | Stage | | | | | | Stage | | |
| | | | | III | | | | | | III | | |
| | | | Monoethyl | (Benzyl | Single | Total | | | Monoethyl | (Benzyl | Single | Total |
| | Assay | pH | Impurity | Indacaterol) | Max | Impurity | Assay | pH | Impurity | Indacaterol) | Max | Impurity |
| Initial | 97.5 | 2.66 | 0.06 | 0.01 | 0.08 | 0.58 | 97.5 | 2.66 | 0.06 | 0.01 | 0.08 | 0.58 |

Stability condition: 40 ± 2° C./75 ± 5% RH

| | | | | | | | | | | | | |
|---|---|---|---|---|---|---|---|---|---|---|---|---|
| 1 M | 101.1 | 2.63 | 0.07 | ND | 0.11 | 0.62 | 98.6 | 2.63 | 0.06 | ND | 0.17 | 0.93 |
| 2 M | 98.1 | 2.63 | 0.05 | ND | 0.07 | 0.51 | 98.3 | 2.62 | 0.06 | 0.02 | 0.56 | 1.69 |
| 3 M | 99.6 | 2.64 | 0.06 | 0.01 | 0.09 | 0.64 | 92.1 | 2.60 | 0.06 | 0.02 | 1.12 | 2.79 |

| | | | | | | | |
|---|---|---|---|---|---|---|---|
| 6 M | NA | 91.1 | 2.62 | 0.08 | 0.04 | 3.16 | 7.58 |

Stability condition: 5 ± 3° C.

| | | | | | | | | | | | | |
|---|---|---|---|---|---|---|---|---|---|---|---|---|
| 1 M | 66.2 | 2.63 | 0.04 | ND | 0.05 | 0.30 | 98.9 | 2.63 | 0.06 | ND | 0.08 | 0.59 |
| 2 M | 18.3 | 2.62 | 0.03 | ND | 0.03 | 0.14 | 100.1 | 2.63 | 0.06 | ND | 0.09 | 0.65 |
| 3 M | 18.7 | 2.61 | 0.03 | ND | 0.03 | 0.14 | 97.2 | 2.61 | 0.06 | ND | 0.09 | 0.71 |

| | | | | | | | |
|---|---|---|---|---|---|---|---|
| 6 M | NA | 98.1 | 2.64 | 0.06 | 0.01 | 0.10 | 0.79 |

Stability condition: 25 ± 2° C./60 ± 5% RH

| | | | | | | | | | | | | |
|---|---|---|---|---|---|---|---|---|---|---|---|---|
| 1 M | 101.5 | 2.64 | 0.06 | ND | 0.09 | 055 | 100.7 | 2.65 | 0.06 | ND | 0.10 | 0.69 |
| 2 M | 99.3 | 2.64 | 0.05 | ND | 0.06 | 0.48 | 101.1 | 2.65 | 0.06 | 0.01 | 0.11 | 0.79 |
| 3 M | 97.0 | 2.63 | NA | NA | NA | NA | 100.9 | 2.64 | NA | NA | NA | NA |

| | | |
|---|---|---|
| 6 M | NA | NA |

Stability condition: 30 ± 2° C./75 ± 5% RH

| | | | | | | | | | | | | |
|---|---|---|---|---|---|---|---|---|---|---|---|---|
| 1 M | 101.1 | 2.63 | 0.06 | ND | 0.07 | 0.57 | 98.7 | 2.64 | 0.06 | ND | 0.11 | 0.83 |
| 2 M | NA | NA | NA | NA | NA | NA | NA | NA | NA | NA | NA | NA |
| 3 M | 98.2 | 2.64 | 0.06 | ND | 0.07 | 0.44 | 95.2 | 2.65 | 0.06 | ND | 0.49 | 1.49 |

| | | | | | | | |
|---|---|---|---|---|---|---|---|
| 6 M | NA | 94.6 | 2.64 | 0.06 | 0.02 | 0.47 | 1.73 |

### D. Indacaterol 100 μg/2 ml Composition with Buffer pH 2.8 (Example 10)

| | Pack | | | | | | | | | | | |
|---|---|---|---|---|---|---|---|---|---|---|---|---|
| | LDPE Respules | | | | | | Amber Glass Vials | | | | | |
| | Test | | | | | | | | | | | |
| | | | | Stage | | | | | | Stage | | |
| | | | | III | | | | | | III | | |
| | | | Monoethyl | (Benzyl | Single | Total | | | Monoethyl | (Benzyl | Single | Total |
| | Assay | pH | Impurity | Indacaterol) | Max | Impurity | Assay | pH | Impurity | Indacaterol) | Max | Impurity |
| Initial | 102.8 | 2.78 | 0.06 | ND | 0.08 | 0.56 | 102.8 | 2.78 | 0.06 | ND | 0.08 | 0.56 |

Stability condition: 40 ± 2° C./75 ± 5% RH

| | | | | | | | | | | | | |
|---|---|---|---|---|---|---|---|---|---|---|---|---|
| 1 M | 102.8 | 2.75 | 0.07 | ND | 0.09 | 0.63 | 102.5 | 2.75 | 0.07 | ND | 0.18 | 1.34 |
| 2 M | 104.2 | 2.74 | 0.06 | ND | 0.08 | 0.57 | 102.0 | 2.74 | 0.06 | 0.02 | 0.50 | 1.60 |
| 3 M | 103.4 | 2.72 | 0.06 | 0.01 | 0.08 | 0.71 | 99.1 | 2.73 | 0.06 | 0.01 | 0.90 | 2.57 |

| | | | | | | | |
|---|---|---|---|---|---|---|---|
| 6 M | NA | 97.4 | 2.72 | 0.08 | 0.03 | 2.43 | 5.44 |

Stability condition: 5 ± 3° C.

| | | | | | | | | | | | | |
|---|---|---|---|---|---|---|---|---|---|---|---|---|
| 1 M | 39.2 | 2.73 | 0.05 | ND | 0.05 | 0.38 | 103.5 | 2.75 | 0.07 | ND | 0.09 | 0.62 |
| 2 M | 23.2 | 2.72 | 0.03 | ND | 0.04 | 0.15 | 104.5 | 2.73 | 0.06 | 0.01 | 0.08 | 0.68 |
| 3 M | 19.5 | 2.71 | 0.04 | ND | 0.04 | 0.19 | 101.2 | 2.71 | 0.06 | ND | 0.09 | 0.66 |

| | | | | | | | |
|---|---|---|---|---|---|---|---|
| 6 M | NA | 98.4 | 2.72 | 0.06 | 0.01 | 0.12 | 0.88 |

Stability condition: 25 ± 2° C./60 ± 5% RH

| | | | | | | | | | | | | |
|---|---|---|---|---|---|---|---|---|---|---|---|---|
| 1 M | 105.5 | 2.78 | 0.07 | ND | 0.09 | 0.59 | 105.0 | 2.76 | 0.06 | ND | 0.10 | 0.69 |
| 2 M | 103.7 | 2.76 | 0.06 | ND | 0.07 | 0.55 | 103.6 | 2.76 | 0.06 | 0.01 | 0.12 | 0.84 |
| 3 M | 101.0 | 2.78 | NA | NA | NA | NA | 99.1 | 2.77 | NA | NA | NA | NA |

| | | |
|---|---|---|
| 6 M | NA | NA |

Stability condition: 30 ± 2° C./75 ± 5% RH

| | | | | | | | | | | | | |
|---|---|---|---|---|---|---|---|---|---|---|---|---|
| 1 M | 105.5 | 2.76 | 0.07 | ND | 0.09 | 0.59 | 104.7 | 2.75 | 0.06 | 0.01 | 0.12 | 0.83 |
| 2 M | NA | NA | NA | NA | NA | NA | NA | NA | NA | NA | NA | NA |
| 3 M | 102.3 | 2.75 | 0.06 | ND | 0.08 | 0.51 | 99.7 | 2.76 | 0.06 | ND | 0.35 | 1.31 |

| | | | | | | | |
|---|---|---|---|---|---|---|---|
| 6 M | NA | 101.4 | 2.75 | 0.06 | 0.05 | 0.51 | 1.95 |

### E. Indacaterol 100 μg/2 ml Composition with 0.01% EDTA and Buffer pH 2.7 (Example 11)

| | Pack | | | | | | | | | | | |
|---|---|---|---|---|---|---|---|---|---|---|---|---|
| | LDPE Respules | | | | | | Amber Glass Vials | | | | | |
| | Test | | | | | | | | | | | |
| | | | | Stage | | | | | | Stage | | |
| | | | | III | | | | | | III | | |
| | | | Monoethyl | (Benzyl | Single | Total | | | Monoethyl | (Benzyl | Single | Total |
| | Assay | pH | Impurity | Indacaterol) | Max | Impurity | Assay | pH | Impurity | Indacaterol) | Max | Impurity |
| Initial | 99.7 | 2.60 | 0.03 | ND | 0.06 | 0.33 | 99.7 | 2.60 | 0.03 | ND | 0.06 | 0.33 |

Stability condition: 40 ± 2° C./75 ± 5% RH

| | | | | | | | | | | | | |
|---|---|---|---|---|---|---|---|---|---|---|---|---|
| 1 M | 98.4 | 2.59 | 0.06 | ND | 0.08 | 0.49 | 98.7 | 2.58 | 0.06 | ND | 0.22 | 0.91 |
| 2 M | 99.9 | 2.58 | 0.05 | ND | 0.07 | 0.46 | 97.7 | 2.59 | 0.06 | ND | 0.35 | 1.24 |
| 3 M | 100.2 | 2.59 | 0.06 | ND | 0.08 | 0.47 | 97.8 | 2.60 | 0.04 | ND | 0.41 | 1.03 |

| | | | | | | | |
|---|---|---|---|---|---|---|---|
| 6 M | NA | 92.2 | 2.61 | 0.07 | ND | 1.92 | 5.61 |

Stability condition: 5 ± 3° C.

| | | | | | | | | | | | | |
|---|---|---|---|---|---|---|---|---|---|---|---|---|
| 1 M | 25.9 | 2.58 | 0.03 | ND | 0.04 | 0.15 | 99.1 | 2.57 | 0.06 | ND | 0.06 | 0.56 |
| 2 M | 69.3 | 2.59 | 0.03 | ND | 0.03 | 0.16 | 98.3 | 2.58 | 0.06 | ND | 0.08 | 0.56 |
| 3 M | 18.0 | 2.60 | 0.03 | ND | 0.04 | 0.16 | 100.3 | 2.58 | 0.06 | ND | 0.06 | 0.56 |

| | | | | | | | |
|---|---|---|---|---|---|---|---|
| 6 M | NA | 2.58 | 0.03 | ND | 0.04 | 0.15 | 99.1 |

Stability condition: 25 ± 2° C./60 ± 5% RH

| | | | | | | | | | | | | |
|---|---|---|---|---|---|---|---|---|---|---|---|---|
| 3 M | 97.0 | 2.58 | NA | NA | NA | NA | 95.3 | 2.59 | NA | NA | NA | NA |

| | | | | | | | |
|---|---|---|---|---|---|---|---|
| 6 M | NA | 97.3 | 2.61 | 0.07 | ND | 0.40 | 1.72 |

Stability condition: 30 ± 2° C./75 ± 5% RH

| | | | | | | | | | | | | |
|---|---|---|---|---|---|---|---|---|---|---|---|---|
| 3 M | 98.0 | 2.58 | 0.07 | ND | 0.08 | 0.55 | 96.7 | 2.58 | 0.06 | ND | 0.24 | 0.93 |

| | | | | | | | |
|---|---|---|---|---|---|---|---|
| 6 M | NA | 97.0 | 2.60 | 0.07 | ND | 0.39 | 1.66 |

### F. Indacaterol 150 μg/2 ml Composition with Buffer pH 2.7 (Example 12)

| | Pack | | | | | | | | | | | |
|---|---|---|---|---|---|---|---|---|---|---|---|---|
| | LDPE Respules | | | | | | Amber Glass Vials | | | | | |
| | Test | | | | | | | | | | | |
| | | | | Stage | | | | | | Stage | | |
| | | | | III | | | | | | III | | |
| | | | Monoethyl | (Benzyl | Single | Total | | | Monoethyl | (Benzyl | Single | Total |
| | Assay | pH | Impurity | Indacaterol) | Max | Impurity | Assay | pH | Impurity | Indacaterol) | Max | Impurity |
| Initial | 98.2 | 2.58 | 0.07 | ND | 0.10 | 0.60 | 98.2 | 2.58 | 0.07 | ND | 0.11 | 0.69 |

Stability condition: 40 ± 2° C./75 ± 5% RH

| | | | | | | | | | | | | |
|---|---|---|---|---|---|---|---|---|---|---|---|---|
| 1 M | 97.4 | 2.57 | 0.06 | ND | 0.10 | 0.59 | 95.7 | 2.56 | 0.07 | ND | 0.25 | 0.99 |
| 2 M | 95.4 | 2.60 | 0.07 | ND | 0.13 | 0.64 | 91.1 | 2.62 | 0.07 | 0.02 | 0.59 | 1.80 |
| 3 M | 96.5 | 2.58 | 0.07 | ND | 0.12 | 0.63 | 90.9 | 2.58 | 0.07 | 0.02 | 0.90 | 2.31 |

| | | | | | | | |
|---|---|---|---|---|---|---|---|
| 6 M | NA | 81.4 | 2.61 | 0.09 | 0.14 | 3.13 | 9.02 |

Stability condition: 5 ± 3° C.

| | | | | | | | | | | | | |
|---|---|---|---|---|---|---|---|---|---|---|---|---|
| 1 M | 77.0 | 2.56 | 0.06 | ND | 0.08 | 0.49 | 97.4 | 2.56 | 0.07 | ND | 0.11 | 0.71 |
| 2 M | 24.3 | 2.65 | 0.07 | ND | 0.12 | 0.64 | 98.5 | 2.57 | 0.07 | ND | 0.09 | 0.66 |
| 3 M | 15.4 | 2.61 | 0.03 | ND | 0.06 | 0.19 | 98.0 | 2.58 | 0.07 | ND | 0.09 | 0.62 |

| | | | | | | | |
|---|---|---|---|---|---|---|---|
| 6 M | NA | 96.9 | 2.61 | 0.07 | ND | 0.14 | 0.67 |

Stability condition: 25 ± 2° C./60 ± 5% Rt

| | | | | | | | | | | | | |
|---|---|---|---|---|---|---|---|---|---|---|---|---|
| 3 M | 95.0 | 2.57 | NA | NA | NA | NA | 92.8 | 2.56 | NA | NA | NA | NA |

| | | | | | | | |
|---|---|---|---|---|---|---|---|
| 6 M | NA | 94.6 | 2.61 | 0.08 | ND | 0.57 | 1.47 |

Stability condition: 30 ± 2° C./75 ± 5% RH

| | | | | | | | | | | | | |
|---|---|---|---|---|---|---|---|---|---|---|---|---|
| 3 M | 96.2 | 2.56 | 0.07 | ND | 0.12 | 0.61 | 94.0 | 2.57 | 0.07 | 0.01 | 0.24 | 0.98 |

| | | | | | | | |
|---|---|---|---|---|---|---|---|
| 6 M | NA | 92.6 | 2.61 | 0.07 | ND | 0.55 | 1.26 |

### Examples 13-15

| | Example No. | | |
|---|---|---|---|
| | 13 | 14 | 15 |
| Indacaterol maleate | 75 | 100 | 125 |
| (Conc. in μg/2 ml) | | | |
| Citric acid monohydrate | 1.85 | 1.85 | 1.85 |
| (% w/v) | | | |
| Trisodium citrate | 0.25 | 0.25 | 0.25 |
| dihydrate (% w/v) | | | |
| Sodium chloride (% w/v) | 0.3 | 0.3 | 0.3 |
| Edetate disodium | 0.01 | 0.01 | 0.01 s |
| (% w/v) | | | |
| Water | q.s 2 ml | q.s 2 ml | q.s 2 ml |

Manufacturing Process
1. Dissolve sodium chloride, edetate disodium, citric acid monohydrate and trisodium citrate dihydrate in water for injection with stirring. Check the clarity of the solution.
2. Add indacaterol maleate to the product of step 1 with stirring.
3. Sonicate the resulting mixture until a clear solution is obtained. Make up the volume with water for injection.
4. Filter the product of step 3 solution through a sterilizing grade filter.
5. Fill the resulting bulk product into LDPE vials.

The contents of the compositions described in Examples 13-15 were poured into the reservoir of a vibrating mesh nebulizer, such as Microbase Device. The compositions were then evaluated using a Next Generation Impactor (NGI) device. The NGI device mimics several components of the respiratory tract.

The data shown below is a representation of the in-vitro aerodynamic particle size distribution (APSD) data by NGI and the Breath simulator (BRS) data for the nebulization compositions of Examples 13-15. FPM refers to fine particle mass. MB refers to mass balance. MOC refers to micro-orifice collector. IP refers to induction port. S1 to S7 refers to stages 1 through stage 7 respectively. DD refers to drug delivery.

In Vitro Data—APSD

Dataset 1

| | Device | IP | S1 | S2 | S3 | S4 | S5 | S6 | S7 | MOC | MB | FPM | DD | % | MMAD | |
|---|---|---|---|---|---|---|---|---|---|---|---|---|---|---|---|---|
| Example | (μg) | (μg) | (μg) | (μg) | (μg) | (μg) | (μg) | (μg) | (μg) | (μg) | (μg) | (μg) | (μg) | FPF | (μm) | GSD |
| 13 | 1.34 | 0.96 | 2.95 | 4.74 | 16.94 | 25.85 | 16.3 | 4.22 | 1.18 | 0.08 | 74.57 | 43.76 | 73.23 | 59.76 | 4.34 | 1.728 |
| 14 | 2.79 | 1.61 | 4.35 | 7.23 | 23.84 | 34.44 | 19.96 | 5.29 | 1.45 | 0.25 | 101.21 | 56.09 | 98.41 | 56.99 | 4.50 | 1.733 |
| 15 | 2.4 | 1.59 | 5.11 | 7.96 | 28.11 | 40.25 | 26.68 | 7.76 | 2.05 | 0.67 | 122.58 | 71.18 | 120.18 | 59.39 | 4.33 | 1.752 |

Dataset 2

| | Device | IP | S1 | S2 | S3 | S4 | S5 | S6 | S7 | MOC | MB | FPM | DD | % | MMAD | |
|---|---|---|---|---|---|---|---|---|---|---|---|---|---|---|---|---|
| Strength | (μg) | (μg) | (μg) | (μg) | (μg) | (μg) | (μg) | (μg) | (μg) | (μg) | (μg) | (μg) | (μg) | FPF | (μm) | GSD |
| 13 | 1.34 | 1.83 | 5.02 | 9.41 | 18.70 | 22.76 | 12.11 | 3.23 | 1.01 | 0.07 | 75.49 | 35.69 | 74.14 | 47.77 | 5.07 | 1.92 |
| 14 | 1.50 | 2.52 | 6.92 | 13.69 | 25.19 | 31.31 | 15.63 | 3.95 | 1.40 | 0.08 | 102.19 | 47.13 | 100.96 | 46.81 | 5.14 | 1.93 |
| 15 | 2.78 | 2.49 | 7.44 | 12.95 | 27.56 | 36.22 | 24.40 | 6.96 | 1.88 | 0.58 | 123.26 | 64.37 | 120.48 | 53.42 | 4.66 | 1.94 |

BRS Data

Dataset 1

| | | | Delivered | Cup | Cup |
|---|---|---|---|---|---|
| | Time | Delivered | dose | Retention | Retention |
| Strength | (mm) | dose (μg) | (%) | (μg) | (%) |
| 13 | 6 min | 38.8 | 51.8 | 3.5 | 4.6 |
| 14 | 6 min | 49.5 | 49.5 | 5.6 | 5.6 |
| 15 | 6 min | 62.8 | 50.3 | 4.8 | 3.9 |

Dataset 2

| | | | Delivered | Cup | Cup |
|---|---|---|---|---|---|
| | Time | Delivered | dose | Retention | Retention |
| Strength | (mm) | dose (μg) | (%) | (μg) | (%) |
| 13 | 7 min | 39.2 | 52.3 | 3.3 | 4.4 |
| 14 | 7 min | 54.1 | 51.1 | 4.9 | 4.9 |
| 15 | 7 min | 64.7 | 51.8 | 5.8 | 4.6 |

1. A nebulization composition comprising
(i) indacaterol or a pharmaceutically acceptable salt thereof;
(ii) an isotonicity agent;
(iii) a buffer; and
(iv) optionally one or more pharmaceutically acceptable excipients selected from the group consisting of a complexing agent, a preservative, a surfactant, and any combination thereof.

2. The nebulization composition according to claim 1, wherein the composition is a solution.

3. The nebulization composition according to claim 1, wherein the composition is a suspension.

4. The nebulization composition according to claim 1, wherein the composition comprises indacaterol maleate.

5. The nebulization composition according to claim 1, wherein the composition comprises micronized indacaterol maleate.

6. The nebulization composition according to claim 1, wherein the composition comprises between about 5 μg/ml and about 1200 μg/ml of indacaterol or a pharmaceutically acceptable salt thereof.

7. The nebulization composition according to claim 1, wherein the volume of the composition is between about 0.1 ml to about 6 ml.

8. The nebulization composition according to claim 1, wherein the composition comprises indacaterol maleate having a mean particle size of about 0.1 micron to about 5 microns.

9. The nebulization composition according to claim 1, wherein the composition has an osmolality of about 200 to about 500 mOsm/kg.

10. The nebulization composition according to claim 1, wherein the composition when administered by a nebulizer provides a mass median aerodynamic diameter of below about 10 microns.

11. The nebulization composition according to claim 1, wherein the composition when administered by a nebulizer provides a geometric standard deviation of below about 5.

12. The nebulization composition according to claim 1, wherein the composition has a geometric standard deviation of about 1 to about 3 and a mass mean aerodynamic diameter of about 4 to about 6 microns when administered by a nebulizer device.

13. The nebulization composition according to claim 1, wherein the composition is a prepackaged, sterile, premixed, premeasured unit dose composition.

14. The nebulization composition according to claim 1, wherein the composition is administered to relieve bronchospasm in a subject.

15. The nebulization composition according to claim 1, wherein the composition is administered once daily.

16. The nebulization composition according to claim 1, wherein the composition is administered twice daily.

17. The nebulization composition according to claim 1, wherein a total daily dose of about 10 μg to about 5000 μg of indacaterol maleate is administered in a single or in divided doses.

18. The nebulization composition according to claim 1, wherein the mean particle size of the indacaterol of a pharmaceutically acceptable salt thereof is from about 0.1 micron to about 5 microns.

19. The nebulization composition according to claim 1, wherein the composition comprises indacaterol maleate having a D₉₀ of not more than 10 microns.

20. The nebulization composition according to claim 1, wherein the composition further comprises a pharmaceutically acceptable surfactant.

21. The nebulization composition according to claim 1, wherein the composition further comprises a pH adjusting agent.

22. The nebulization composition according to claim 1, wherein the composition further comprises a complexing agent.

23. The nebulization composition according to claim 1, wherein the composition further comprises a pharmaceutically acceptable preservative.

24. The nebulization composition according to claim 1, wherein the pH of the composition is between about 2 and about 8, such as between about 2.0 and about 4.0.

25. The nebulization composition according to claim 1, wherein the composition contains not more than about 0.1% of a monoethyl impurity of indacaterol.

26. The nebulization composition according to claim 1, wherein the composition contains not more than about 0.05% of a benzyl Indacaterol impurity.

27. The nebulization composition according to claim 1, wherein the composition is contained in a prefilled container.

28. A nebulization composition comprising:
(i) indacaterol or a pharmaceutically acceptable salt thereof,
(ii) sodium chloride,
(iii) tartaric acid and monosodium citrate, and
(iv) water for injection.

29. A nebulization composition comprising:
(i) indacaterol or a pharmaceutically acceptable salt thereof,
(ii) sodium chloride,
(iii) tartaric acid and sodium tartrate dihydrate, and
(iv) water for injection.

30. A nebulization composition comprising:
(i) indacaterol or a pharmaceutically acceptable salt thereof,
(ii) an isotonicity agent,
(iii) a pH adjusting agent, and
(iv) optionally a pharmaceutically acceptable excipient selected from the group consisting of a complexing agent, a preservative, and any combination thereof; and
(vi) water for injection.

31. The nebulization composition according to claim 30, wherein the composition comprises indacaterol or a pharmaceutically acceptable salt thereof, sodium chloride, a pH adjusting agent, and water for injection.

32. A kit for the treatment, prevention or amelioration or one or more symptoms of a disease or disorder associated with bronchoconstriction, comprising
(i) a nebulizer; and
(ii) a nebulization composition according to claim 1.

33. The kit according to claim 32, wherein the nebulizer is selected from a jet nebulizer, ultrasonic nebulizer, vibrating mesh nebulizer and a breath actuated nebulizer.

34. A method of treating relief of bronchospasm in a subject suffering from asthma or chronic obstructive pulmonary disorder comprising administering a nebulizable composition according to claim 1.
